(19) United States
(12) Patent Application Publication
(10) Pub. No.: US 2024/0252082 A1
(43) Pub. Date: Aug. 1, 2024

(54) METHOD AND SYSTEM FOR PROVIDING A PLURALITY OF CONSECUTIVE TESTS FOR A TEST PERSON

(71) Applicant: Hoffmann-La Roche Inc., Little Falls, NJ (US)

(72) Inventors: Kirsten Ingrid TAYLOR MONSCH, Basel (CH); Werner Louis Paul POPP, Basel (CH); Alessandra Elena THOMANN, Basel (CH); Ronald Benjamin POSTUMA, Quebec (CA)

(73) Assignee: Hoffmann-La Roche Inc., Little Falls, NJ (US)

(21) Appl. No.: 18/590,571

(22) Filed: Feb. 28, 2024

Related U.S. Application Data
(63) Continuation of application No. PCT/EP2022/073090, filed on Aug. 18, 2022.

(30) Foreign Application Priority Data
Aug. 31, 2021 (EP) 21194112.5

(51) Int. Cl. A61B 5/16; A61B 5/00

(52) U.S. Cl. CPC A61B 5/162; A61B 5/7435; A61B 5/7475; A61B 5/6898

(57) ABSTRACT

The present disclosure relates to a computer-implemented method of providing a plurality of consecutive tests for a test person comprises the steps of:
providing at least one set of visual stimuli having a predefined order, wherein in a test the test person is expected to select the stimuli according to their predefined order, such that the response to a stimulus is a function of the response to the preceding stimulus according to the predefined order,
providing a pattern defined by a plurality of predetermined positions,
for each of the plurality of tests, mapping the stimuli to the pattern in a unique sequence according to a pseudo-random distribution function,
wherein said function is defined:
   such that a mean distance between each pair of consecutive pattern positions, to which a stimulus and its preceding stimulus are mapped, is within a first predetermined range for each of the plurality of tests, and/or
   such that a total distance is within a second predetermined range for each of the plurality of tests, the total distance summing the distances between each pair of consecutive pattern positions to which a stimulus and its preceding stimulus are mapped.

### TECHNICAL FIELD

The present application generally relates to the field of neuropsychological test methods and systems. An example of such a test is the Trail Making Test (TMT).

### BACKGROUND

Neuropsychological tests can be used as diagnostic and disease monitoring tools in clinical and research settings. An exemplary test purpose may be to quantify cognitive dysfunction resulting from brain damage or disease and its changes over time. An example of such a test comprises the Trail Making Test (TMT).

The Trail Making Test (TMT) presents individuals with a pencil and a single piece of paper circled numbers (TMT-A), followed by a single piece of paper with circled numbers and letters (TMT-B). For TMT-A, individuals are asked to place their pencil on the circle with the numeral 1 and then, without removing the pencil from the paper, to draw lines to circled numbers in ascending order, as quickly and accurately as possible. Mistakes are manually noted by the tester. The TMT-B is administered in an identical fashion with the exception that individuals are instructed to switch between ascending numbers and letters. Both the TMT-A and TMT-B are preceded by short practice tests, and in both forms, the beginning and end stimuli are demarcated. The total time required to complete the TMT-A and TMT-B may be noted by the examiner, as well as the number of mistakes. One common outcome measure for the TMT is TMT-B/TMT-A (i.e. the time required to complete TMT B divided by the time required to complete TMT A), which aims to quantify task-switching (executive function) ability while controlling for baseline visuo-motor and low-level cognitive processing speed.

It is further known to use electronic devices to administer neuropsychological tests. For example, Dahmen et.al. proposes a digital version of a Trail Making Test (TMT), cf. Dahmen et.al.: An Analysis of a Digital Variant of the Trail Making Test Using Machine Learning Techniques, Technol Health Care. 2017; 25(2): 251-264, doi: 10.3233/THC-161274. The work discloses a digital, mobile variant of the original paper-based TMT. Rather than creating an entirely new cognitive test, the digital TMT (dTMT) is designed to look as similar to the original test as possible in order to establish initial convergent validity with the paper-based version. The test is administered using a capacitive touchscreen tablet. Like in the paper version of TMT, in the dTMT a test person is requested to draw a line on the screen tablet. Also fewer stimuli may be used than in the paper version (i.e. 20 vs. 25, respectively). However, in the dTMT there appears the risk that the level of difficulty may vary across the time, i.e. across several test session. Accordingly, the dTMT does not seem to provide well-balanced TMT ‘alternate forms’ for longitudinal assessment, which controls for task difficulty and therefore confounds on primary TMT outcome measures.

The daily use of electronic communication devices, in particular smartphones, has become common for a large percentage of the population including older people. However, smart phones usually have significantly smaller screens compared to a tablet screen or the size of a piece of paper (e.g. A4 format). Moreover, smartphones are usually controlled by touch gestures, in particular by tap gestures. The use of for example an electronic pencil with a smartphone is however less common and less comfortable, as it would require two hands (one for holding the smartphone and one for holding the electronic pencil). For these reasons, the Trail Making Test (TMT), e.g. in the form of the dTMT developed by Dahmen et.al, does not appear to be suitable to be implemented on a smart phone.

### SUMMARY

A simplified summary of some embodiments of the disclosure are provided in the following to give a basic understanding of these embodiments and their advantages. Further embodiments and technical details are described in the detailed description presented below.

According to an embodiment, a computer-implemented method of providing a plurality of consecutive tests for a test person comprises the steps of:
providing at least one set of visual stimuli having a predefined order, wherein in a test the test person is expected to select the stimuli according to their predefined order (e.g. “1”, “2”, . . . ), such that the (expected) response to a (current) stimulus (e.g. “2”) may be a function of the response to the preceding stimulus (e.g. “1”) according to the predefined order,
providing a pattern defined by a plurality of predetermined positions,
for each of the plurality of tests, mapping the stimuli to the pattern in a unique sequence according to a pseudo-random distribution function,
wherein said function is defined:
   such that a mean distance between each pair of consecutive pattern positions, to which a stimulus and its preceding stimulus are mapped, is within a first predetermined range for each of the plurality of tests, and/or
   such that a total distance is within a second predetermined range for each of the plurality of tests, the total distance summing the distances between each pair of consecutive pattern positions to which a stimulus and its preceding stimulus are mapped.

In other words, for each of the plurality of tests, each stimulus of the set may be mapped to a different position in the graphical pattern according to the pseudo-random distribution function.

The tests may be cognitive tests. In particular, the tests may for example be based on the principles of a Trail Making Test (TMT), i.e. a TMT-A and a TMT-B, or a plurality of TMTs.

By providing such a method the test difficulty can be controlled across the plurality of tests administered over time. In other words, the method ensures comparable levels of test difficulty when testing repeatedly over time, thereby increasing the confidence in ascribing potential differences in test performance over time to changes in brain function (e.g. progressive neurodegeneration, therapeutic effects of novel drug) and not the confounding effects of test difficulty. The matching of test difficulty is particularly advantageous the test is presented on a user interface with a small display, for example a smartphone. Small displays may lead to a reduced number of presentable stimuli and thus may lead to an increased risk of clustering of consecutive stimuli thereby reducing task difficulty as measured by time to completion. The present method provides an optimized user interface by ensuring comparable levels of test difficulty despite a potentially small display size. In addition, by providing tests with a predetermined test pattern (i.e. a pattern defined by a plurality of predetermined positions), the space between positions may be constant.

A position may be understood as a predefined location on a screen.

The pattern may be a graphical pattern.

The pattern may have a grid form.

The pattern may be configured such that it fits to a display screen. The pattern may have for example n*m (e.g. 15) positions, n and m being integers.

The distance between each pair of consecutive pattern positions, to which a stimulus and its preceding stimulus are mapped in the pattern having a grid form, may be defined by the number of horizontal and/or vertical steps in the grid between the pair. For example, the number of required steps between two horizontally adjacent or two vertically adjacent positions may be one. The distance between two diagonally adjacent positions (i.e. where one vertical and one horizontal step is required to get from one position to the other) may be two.

The stimuli may be mapped onto the pattern in a pseudo-randomized sequence. The predefined order may be a pseudo-random and/or sequential order.

The mean distance may be an arithmetic mean step count.

The pseudo-random distribution function may comprise at least one rule which is defined such that clusters of consecutive stimuli on the pattern are reduced.

For example, a cluster of consecutive stimuli “1-2-3 . . . ” placed in adjacent positions on the screen can be avoided in this way.

The stimuli may be mapped to the pattern in a pseudo-randomized sequence. The same pseudo-random distribution function may be used for all tests. For example, the same function (e.g. the same distribution rules) may be used for every test. However, the distribution result of the function may desirably be different for every test. It is thus referred to above to a “unique sequence” (i.e. a different sequence for each test).

The pattern may be identical for all tests.

The stimuli may comprise numerical or alphanumerical stimuli or other signs. In other words, the stimuli may generally comprise symbols, for example letters or numbers. Examples of further possible signs comprise non-latin letters, e.g. Chinese signs, whereby arabic digits may still be used in a respective TMT (e.g. TMT A: Arabic digits; TMT B: Arabic digits, numbers in Traditional Chinese). Still further examples comprise Hebrew, Greek, and Arabic signs.

The number of positions of the pattern may correspond to the number of stimuli of one set of stimuli.

An individual test (e.g. TMT-A-type test) may comprise two consecutive test parts. At least one set of stimuli may comprise a first set for the first test part and a second set for the second test part. The stimuli of the second set may follow the stimuli of the first set according to the predefined order of the first set. In the first test part the first set may be mapped to the positions of the pattern, and consecutively in the second test part the second set may be mapped to the positions of the pattern. The number of stimuli of each test part may correspond to the number of pattern positions.

Accordingly, in case of a TMT, the set of stimuli may generally correspond to a trial, i.e. a single screen test or test page. In other words, a trial or single screen test or test page may be understood as a test whose stimuli may fit all together on a single screen (e.g. a set with 15 stimuli which fit on the screen).

However, the number of sets of stimuli may also be any larger number, in particular n-times the number sets with n being an integer. As described above, the number of sets of stimuli may for example be two (referred to as two test parts in the following), such that a test as provided by the set of stimuli fits on two patterns and accordingly on two screens. Said screens may be presented consecutively to the test person, for example by the same display device. The test comprising two consecutive test parts may be referred to as a diad, as further described in context of the figures.

As a consequence, due to the reduced number of presented stimuli which can comfortably fit a smartphone screen, two screens may be presented for each test (i.e. each diad).

The plurality of tests may comprise a first and a second type of test which are provided one after the other in the same testing session. The at least one set of stimuli may comprise a first set or a first and second set for the first type of test and a third set or a third and fourth set for the second type of test.

In case of a test designed on the principles of a TMT, the first type may follow the principles of a TMT-A, and the second type may follow the principles of a TMT-B. The first and the second type of test may form a session together. Each of the test types may comprise two test parts. The first test type may comprise a first and second set (i.e. a diad), and second test type may comprise a third and fourth set (i.e. a further diad). A session may be provided to a test person in one sitting, e.g. without any longer interruptions between the tests and/or on the same day. The sessions may be repeated periodically, e.g. every month, but with different stimuli distributions due to the pseudo-random distribution function.

The pseudo-random distribution function may be defined such that the mean distance of consecutive stimuli within a couple of first and second test types (i.e. in a session) on the pattern may be the same or within a third predetermined range being smaller than the first predetermined range.

The method may further comprise the step of presenting, for each individual test, the stimuli mapped to the positions of the pattern on a display or a touchscreen. The terms “display” and “touchscreen” desirably refer to devices, meanwhile the term screen may rather refer to the visual presentation provided by said device, or in other words, the visual area of said device.

The method may further comprise the step of calculating a test result as a function of the test person's stimulus responses. In other words, the test result may be calculated as a function of the touchscreen responses (i.e. the test person's gestures in response to the presented stimuli).

The stimulus responses may be tap responses on the screen for selecting a stimulus.

The test result may be calculated as a function of whether the test person has selected the stimuli according to their correct predefined order and/or as a function of the test person's response time.

The method may further comprise the step of highlighting the most recently correctly selected stimulus, or the last two correctly selected stimuli, in a first predefined form.

The method may further comprise the step of, in case the test person selects an incorrect stimulus, highlighting the incorrectly selected stimulus in a second predefined form.

In a further embodiment, a computer program comprises computer-readable instructions which when executed by a data processing system cause the data processing system to carry out the method according to any one of preceding methods.

In a further embodiment, a recording medium readable by a computer and having recorded thereon a computer program includes instructions for executing the steps of a method according to any one of the preceding methods.

In a further embodiment, a processing device for providing a plurality of consecutive tests for a test person, is configured to:
provide at least one set of visual stimuli having a predefined order, wherein in a test the test person is expected to select the stimuli according to their predefined order, such that the response to a stimulus is a function of the response to the preceding stimulus according to the predefined order,
provide a pattern defined by a plurality of predetermined positions,
for each of the plurality of tests, map the stimuli to the pattern in a unique sequence according to a pseudo-random distribution function,
wherein said function is defined:
   such that a mean distance between each pair of consecutive pattern positions, to which a stimulus and its preceding stimulus are mapped, is within a first predetermined range for each of the plurality of tests, and/or
   such that a total distance is within a second predetermined range for each of the plurality of tests, the total distance summing the distances between each pair of consecutive pattern positions to which a stimulus and its preceding stimulus are mapped.

The present technology solves the problem of providing a plurality of tests with a comparable test difficulty. In particular, due to the proposed pseudo random distribution function, the probability can be reduced that differences in task performance of a test person are due to sequence difficulty. Accordingly, the test may also be provided in a relatively simple pattern, as for example a grid-like pattern with only a few predetermined positions (for example 15). As a consequence, the test may also be presented on a relatively small electronic display, for example on a smartphone, and/or responses may be collected by means of tap gestures.

The foregoing general description and the following detailed description are exemplary and explanatory only and are not restrictive of the invention as claimed.

### BRIEF DESCRIPTION OF THE DRAWINGS

The foregoing summary as well as the following detailed description of preferred embodiments are better understood when read in conjunction with the appended drawings. For illustrating the invention, the drawings show exemplary details of systems, methods, and experimental data. The information shown in the drawings are exemplary and explanatory only and are not restrictive of the invention as claimed. In the drawings:

FIG. 1 shows a flowchart computer-implemented method for providing a plurality of consecutive tests;

FIG. 2 shows a block diagram of a processing device for providing a plurality of consecutive tests and an optional display device;

FIG. 3a shows an exemplary pattern defined by a plurality of predetermined positions;

FIG. 3b shows position labels of the pattern of FIG. 3a;

FIG. 4 shows exemplary screenshots of a first test (here according to the test-type TMT-A) implemented on a smartphone or another electronic device;

FIG. 5 shows exemplary screenshots of a second test (here according to the test-type TMT-B) implemented on a smartphone or another electronic device.

### DETAILED DESCRIPTION

The present disclosure relates to computer-implemented methods and systems for providing a plurality of consecutive tests to a test person. The method may be implemented on a smartphone or another user interface and/or electronic device.

The tests may be neuropsychological tests. Such a test may be used to support diagnostic workups in clinical settings and/or track changes in cognitive functioning over time. An exemplary test purpose may be to assess cognitive dysfunctions resulting from brain damage or disease and their changes over time. An example of such a test comprises the Trail Making Test (TMT).

As explained in more detail in the following and with reference to the figures, the design of the tests provided by the method may make the execution of for example a digital test based on the principles of the TMT hypothetically feasible for a wide range of patients with motor impairments (e.g. patients with severe tremor) that may otherwise confound test outcome measures. A test person may respond to the test by tapping the stimuli sequentially instead of drawing a line. Such stimulus responses (i.e. touchscreen responses) are easier for users with dexterity impairments. A plurality of tests may be developed such that test difficulty is counterbalanced over each testing session. The tests may also be adaptable for other languages and symbols, e.g. eastern languages using non-Latin alphabets, e.g., Polish. Moreover, a specific error handling may be implemented.

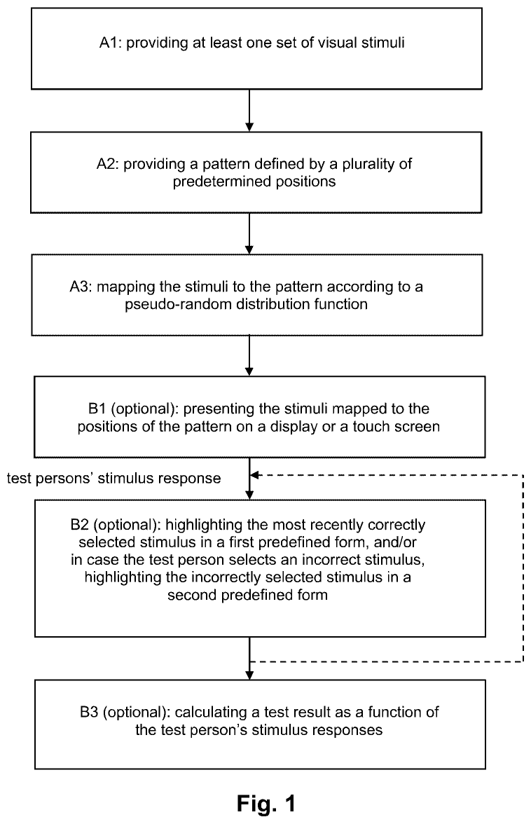

FIG. 1 shows a flowchart computer-implemented method for providing a plurality of consecutive tests for a test person.

In a step A1, at least one set of visual stimuli having a predefined order is provided. In a test, or in particular in each test, the test person is expected to select the stimuli according to their predefined order, such that the response to a stimulus is a function of the correct response to the preceding stimulus according to the predefined order.

In a step A2 a pattern is provided which is defined by (or which defines or comprises) a plurality of predetermined positions. Steps A1 and A2 may be carried out simultaneously or consecutively, e.g. step A1 before step A2 or vice-versa.

In Step A3 the stimuli are mapped to the pattern in a unique sequence according to a pseudo random distribution function. This may be done for each of the plurality of tests. The function is defined such that a mean distance between each pair of consecutive pattern positions, to which a stimulus and its preceding stimulus are mapped, is within a first predetermined range for each of the plurality of tests. Additionally or alternatively the function is defined such that a total distance is within a second predetermined range for each of the plurality of tests, the total distance summing the distances between each pair of consecutive pattern positions to which a stimulus and its preceding stimulus are mapped. Accordingly, step A3 may result in a set of tests mapped to the same pattern but in different sequences according to the pseudo random distribution function.

Steps A1 to A3 may also be carried out in advance to the subsequent optional steps. For example, the resulting set of mapped tests may be stored (for example centralized or decentralized) and be provided only at a later time to one or several user interfaces and/or electronic devices (for example smartphones) on which the tests may be carried out.

In optional step B1 for each individual tests the stimuli mapped to the positions of the pattern are presented on a display or a touchscreen device, in particular to a test person. More in particular, the mapped tests may be presented consecutively on the same display or a touchscreen device. The display and/or touchscreen device may be part of a user interface and/or electronic device (for example a smartphone). As mentioned, the tests may also be presented repeatedly over time, for example one test or a subset of tests at one day per month. In this way it is possible to monitor the evolution of a test person's performance over time to infer changes in brain function. This is in particular possible, since the tests have a balanced test difficulty.

Once a test is presented on the display or a touchscreen device, a test person can respond to it. In other words, the test person's stimulus response may be received. In one example, as further described in more detail in context of FIGS. 3 to 5, a test person may respond to the stimuli provided by a test by selecting the stimuli according to their predefined order (for example, first stimulus “1”, then stimulus “2, etc.). More in particular, the stimulus responses may comprise gestures of the test person, for example tap gestures on a touchscreen.

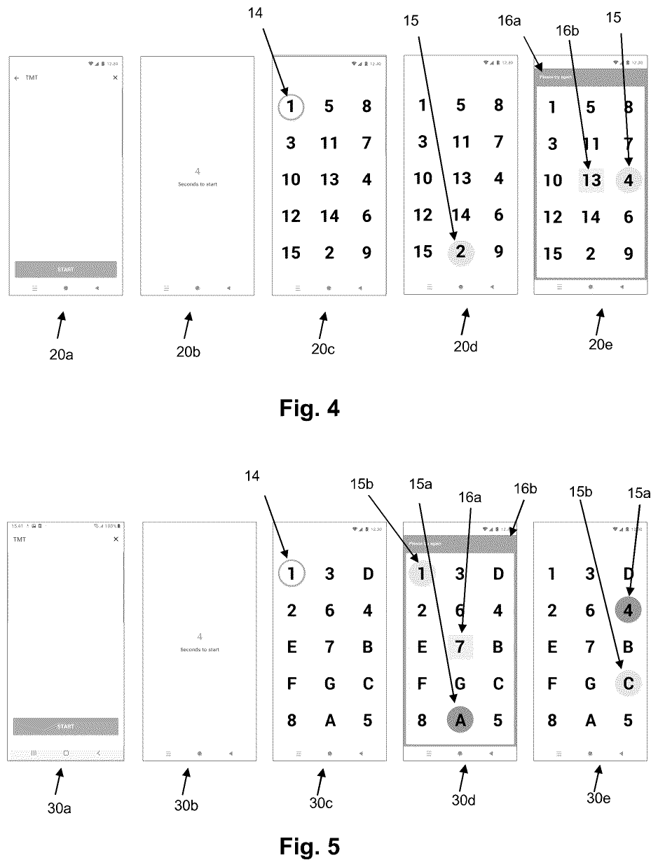

In optional step B2, based on each received stimulus response, it is determined whether the response is correct or incorrect. For example, in case the most recently correctly selected stimulus response was “1”, the presently expected response comprises a selection of the stimulus “2”. In this regard the most recently (i.e. the preceding) correctly selected stimulus or the last two correctly selected stimuli may be highlighted in a first predefined form. This may assist the test person in recognizing how far he/she has already advanced in the current test. Moreover, in case the test person selects an incorrect stimulus, the incorrectly selected stimulus may be highlighted in a second predefined form. Examples of the first and second highlighting form are described in context of FIGS. 4 and 5.

Optional step B2 may be repeated until each stimulus of the present test has been correctly responded to by the test person. In other words, optional step B2 may be repeated until each stimulus has been correctly selected by the test person according to their predefined order.

The method may comprise at least one of the following termination conditions:
1. The test has been correctly been completed;
2. A predefined number of consecutive errors (e.g. three errors) have been made by the user, e.g. by consecutively selecting incorrect stimuli instead of the expected (correct) stimulus;
3. A predefined total number of errors (e.g. ten errors) have been made by the user in a TMT-A diad or a TMT-B diad, e.g. by selecting incorrect stimuli instead of the expected (correct) stimuli;
4. A predefined amount of time (e.g. 120 sec) of inactivity has been passed, where e.g. no stimulus has been selected, at all.

In optional step B3 a test result is calculated as a function of the test person's stimulus responses. In other words, the test result may be calculated as a function of the test person's gestures in response to the presented stimuli. The result may be calculated for example as a function of the time to successfully pass the test and may optionally take into account the number of incorrectly selected stimuli.

According to further examples, the test result may be calculated as a function of at least one of:
The total time to complete a predetermined number of tests, e.g. a predetermined number of tests as for instance one diad (two screens)
The average time to complete a predetermined number of tests, e.g. a predetermined number of tests as for instance one diad (two screens)
The total time to complete a predetermined number of tests (e.g. a predetermined number of tests as for instance one diad) divided by total step count
The average time to complete a predetermined number of tests (e.g. a predetermined number of tests as for instance one diad) divided by median step count
The total time to complete a predetermined number of tests (e.g. a predetermined number of tests as for instance one diad) divided by total step count
The number of errors (i.e., non-sequential responses)
The TMT-B/TMT-A ratios of all the above
Features of motor symptoms (e.g. tremor) measures based on IMU sensor measurements and/or touchscreen measurements.

The set of tests may in particular be based on the principles of the Trail Making Tests (TMT). The TMT may comprise a test based on TMT-A principles, and a test based on TMT-B principles. The TMT-A-type test desirably only comprises stimuli in the form of numbers, meanwhile the TMT-B-type test desirably comprises numbers and letters. Each of the TMT-A-type and TMT-B-type tests may also be referred to as a diad. Each diad may comprise two trials. A trial or single screen test may be understood as a test whose stimuli may fit all together on a single screen (e.g. a set with 15 stimuli which fit on the screen, as shown in screenshots 20c to 20e of FIG. 4 and screenshots 30c to 30e of FIG. 5).

Diads of one TMT-A-type and one TMT-B-type test may form together a session. A series of TMT-A and TMT-B tests administered over time may comprise a plurality of sessions. Sessions may be provided to a test person in regular periods, e.g. every month during e.g. 2 years.

The following table 1 provides one example of a pseudo-randomly mapped set of stimuli to the grid pattern, wherein the “StimulusCode” indicates the grid position on the screen (cf. the position labels indicated in FIG. 3a), trial refers to screens 1 and 2 (diad), and the mapped stimuli define which stimulus (numbers (TMT-A-type) and numbers and letters (TMT-B-type)) appear in which grid location (StimulusCode).

TABLE 1
Example of pseudo-randomly mapped TMT-A- and TMT-
B-type stimuli to screen position (StimulusCode).

| | Trial | Mapped TMT-A-type | Mapped TMT-B-type |
|---|---|---|---|
| StimulusCode | (screen) | stimulus | stimulus |
| 1 | 1 | 1 | 1 |
| 2 | 1 | 4 | 2 |
| 3 | 1 | 7 | 3 |
| 4 | 1 | 5 | A |
| 5 | 1 | 2 | E |
| 6 | 1 | 11 | 5 |
| 7 | 1 | 10 | B |
| 8 | 1 | 14 | G |
| 9 | 1 | 6 | F |
| 10 | 1 | 8 | 8 |
| 11 | 1 | 15 | 4 |
| 12 | 1 | 12 | 7 |
| 13 | 1 | 3 | 6 |
| 14 | 1 | 13 | D |
| 15 | 1 | 9 | C |
| 1 | 2 | 1 | H |
| 2 | 2 | 5 | 11 |
| 3 | 2 | 8 | L |
| 4 | 2 | 6 | 13 |
| 5 | 2 | 11 | O |
| 6 | 2 | 14 | 14 |
| 7 | 2 | 4 | 9 |
| 8 | 2 | 10 | N |
| 9 | 2 | 12 | M |
| 10 | 2 | 13 | K |
| 11 | 2 | 15 | 10 |
| 12 | 2 | 3 | 15 |
| 13 | 2 | 9 | 12 |
| 14 | 2 | 2 | I |
| 15 | 2 | 7 | J |

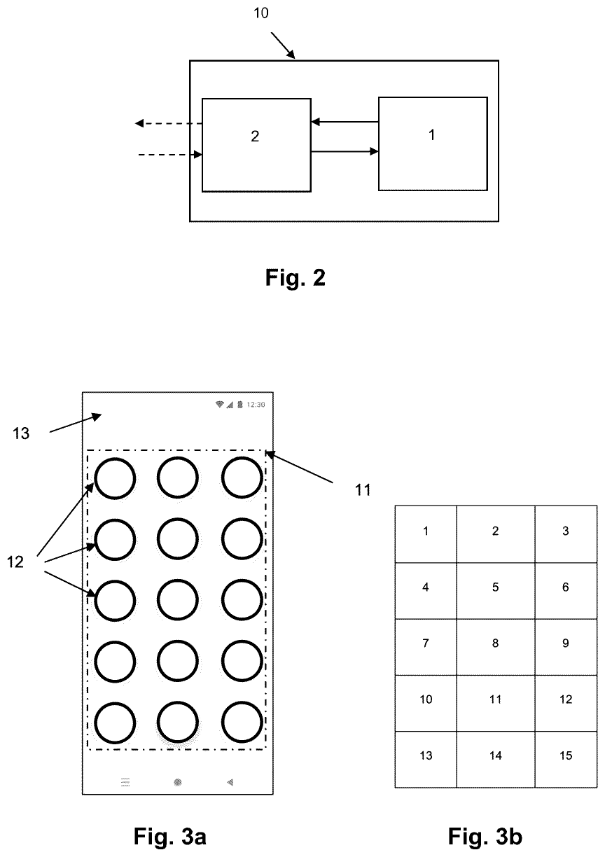

FIG. 2 shows a block diagram of a processing device 1 for providing a plurality of consecutive tests and an optional display device 2. The devices 1 and 2 may be comprised of an electronic device 10, for example a smartphone. Other examples comprise a personal computer or a tablet computer. The processing device 1 and the optional display device 2 may however also be remote to each other. For example, the processing device 1 may be (or may part of or may comprise) a centralized server or part of a cloud application, and the display device 2 may be part of a client device, for example a smartphone, tablet computer or personal computer.

The processing device may comprise a processor, for example CPU and/or a GPU, and optionally a memory. More generally, the processing device may comprise digital circuits, computer-readable storage media, as one or more computer programs, or a combination of one or more of the foregoing. The computer-readable storage media may be non-transitory, e.g., as one or more instructions executable by a cloud computing platform and stored on a tangible storage device.

The display device may be touch sensitive (i.e. a touchscreen device) or may be without a touch function. In the latter case, the electronic device 10 may comprise further input means, for example a mouse or touchpad. The electronic device and/or the display may constitute a user interface. The processing device 1 may be configured to carry out the method according to the present disclosure, as for example described in context of FIG. 1. The tests mapped to the pattern according to the pseudo-random distribution function may be consecutively transmitted to the display device 2 for presentation purposes. The test person's stimulus responses received by the display device (or another input means) may be transmitted back to the processing device 1. In response, in particular once all stimulus responses for one test are received, the processing device 1 may calculate the test result.

The method may be implemented as a computer program comprising computer-readable instructions which when executed by processing device 1 cause the processing device 1 to carry out the method according to the present disclosure. However, the method according to the present disclosure may also be carried out by an external processing device (not shown in FIG. 2), i.e. remote to the electronic device 10 (e.g. a smartphone). For example, the output of step A3 (i.e. the mapped stimuli) may be provided to then to the electronic device 10. The (internal) processing device 1 may then use this (predetermined) data to carry out the further steps B1 and B2. Step B3 may be carried out by any of the internal and external processing devices.

In particular, the computer program may comprise a pseudo-random distribution function, as described above. Accordingly, the order of stimulus placement on a screen may be pseudo-randomized to ensure that consecutive stimuli are not e.g. clustered together on the screen or involve repetitive correct responses (e.g. up-down-up-down), that the location of a specific stimulus is not overly repetitive across the tests, and that movements from one stimulus to next on the screen are not overly repetitive across the tests.

The pseudo-random distribution function may be defined such that a mean distance between each pair of consecutive pattern positions, to which a stimulus and its preceding stimulus are mapped, is within a first predetermined range for each of the plurality of tests.

For example, in case of e.g. the test setting of FIG. 4, the median step count may be 2.56 in the grid-form pattern between consecutive stimuli (i.e. between each pair of consecutive pattern positions). The first predetermined range may be for example 2.5±0.25 (i.e., 2.25-2.75).

The pseudo-random distribution function may also be defined such that a total distance is within a second predetermined range for each of the plurality of tests, the total distance summing the distances between each pair of consecutive pattern positions to which a stimulus and its preceding stimulus are mapped.

For example, in case of e.g. the test setting of FIGS. 4 and 5, the mean total number of steps on the two screens may be 74 in the grid-form pattern summing the distances between all consecutive stimuli (i.e. between each pair of consecutive pattern positions). The second predetermined range may be for example be (summed over 2 trials) 38*2=76±3 (i.e., 73-79).

Moreover, the pseudo-random distribution function may be defined such that the distance metrics for the TMT-A screens and TMT-B screens presented within a given testing session may be the same or within a third predetermined range being smaller than the first predetermined range. Accordingly, TMT-A and TMT-B diads may be pairwise matched according to the exact same total number of steps and exact same mean median number of steps, to optimize the normalized performance metric.

Moreover, the pseudo-random distribution function may be defined such that the distance metrics for different TMT-A screens presented over time, and for different TMT-B screens presented over time, are within a fourth predetermined range, to optimize the normalized performance metric over repeated administrations (e.g. in a longitudinal behavioral study lasting several years with monthly testing sessions). The fourth predetermined range may smaller than the first predetermined range. Accordingly it may be comparable to the third predetermined range (or alternatively may be larger than the third predetermined range).

The pseudo-random distribution function comprises at least one rule which is defined such that clusters of consecutive stimuli on the pattern are reduced. Exemplary rules are described in the following, which may be applied in any combination in the function.

According to a first exemplary rule, two consecutive stimuli in ascending order may appear only once (or not at all) in a trial. An example would be that in TMT-A the screen with a grid pattern with three columns (cf. for example the screen format of FIG. 4) comprises in one row the stimuli “1-2-5”, where consecutive stimuli “1” and “2” are placed in ascending order on the pattern.

According to a second exemplary rule, two consecutive stimuli in descending order may appear only once (or not at all) in a trial. An example would be that in TMT-A the screen with a grid pattern with three columns (cf. for example the screen format of FIG. 4) comprises in one row the stimuli “1-5-4”, where consecutive stimuli “4” and “5” are placed in descending order on the pattern.

According to a third exemplary rule, it may be ensured that trials with one ascending and one descending sequence have at least one pair split over rows. Accordingly, if there is one ascending and one descending sequence, the rule may define to split one of the two sequences by a row. An example would be that in the screen with a grid pattern with three columns (cf. for example the screen format of FIG. 4) comprises in a first row the stimuli “1-2-5” and in a second row below the stimuli “4-9-12”, where consecutive stimuli “5” and “4” are split by one row.

According to a fourth exemplary rule, the same direction of stimulus response on consecutive trials (e.g. up, down, left, right, up-left, up-right, down-left, and/or down-right) may appear only once (or not at all) but not more than once in succession during a trial. An example would be that in the screen with a grid pattern with five columns (cf. for example the screen format of FIG. 3a) comprises the following five rows of stimuli (where X represents any unique numeral between 1-15 not represented in the row) “1 X X”, “8 X X”, “2 X X”, “7 X X”, “3 X X”, where consecutive responses “1-2” and “2-3” have the same direction.

According to a fifth exemplary rule, the same direction of stimulus response (e.g. up, down, left, right, up-left, up-right, down-left, and/or down-right) for consecutive alternating responses trials (e.g. up-down-up), which may appear a maximum twice in a trial. An example would be that in the screen with a grid pattern with three columns (cf. for example the screen format of FIG. 4) comprises in a first row with the stimuli “1-7-2”, and in a second row with the stimuli “3-8-4” where alternating responses “1-2” and “3-4” both have the same direction.

FIG. 3a shows an exemplary pattern (in FIG. 3a indicated by “11”) defined by a plurality of predetermined positions 12. FIG. 3b shows position labels of the pattern of FIG. 3a. In the shown example, the pattern may be presented on a smartphone screen 13 or another screen with a similar format. The screen shown in FIGS. 4 and 5 may correspond to that one of FIG. 3a and may use the pattern 11. The pattern may be used for each of the tests of the test set. In other words, the tests may merely differ by the order of the stimuli mapped to the pattern positions (and optionally by the stimuli themselves). Said order may be a pseudo-randomized order, i.e. it may be determined by the pseudo-random distribution function.

The pattern may have a grid-like form. In this way the pattern may be fitted to rectangular screens. The pattern may however also have any other form, for example a circular or spiral form. In particular, the pattern may comprise n*m pattern positions 12. The pattern format may be such that n is smaller than m. In this way, the pattern may be fitted to wide screen devices, as for example used in smart phones. In the shown example, the pattern comprises (nm) 3*5 pattern positions=15 pattern positions in total. Accordingly, the number of provided pattern positions may be relatively small, for example between 10 and 20. The pattern positions may be ordered as indicated by the labels shown in FIG. 3b.

Hence, a test mapped to the pattern may still show relatively large stimuli, which are thus well visible and easily selectable for example by tap gestures with a finger. The test may hence be also suitable to be carried out by older people or generally any people with a poor eyesight and/or who are not used to handle smartphones. Moreover, since the pattern positions are predetermined, the graphical test format becomes rapidly intuitive, in particular when a regular pattern form is used as e.g. a grid form. The test person may hence focus on the test itself, i.e. the test stimuli and is not distracted by any pattern variations. Finally, the predetermined pattern positions for the plurality of tests also help to ensure comparable levels of test difficulty.

FIG. 4 shows exemplary screenshots 20a to 20e of a first test implemented on a smartphone or another electronic device. The screenshots may be consecutive, wherein only some but not all consecutive screenshots of the test are shown. The test shown in FIG. 4, in particular in screenshots 20c to 20e, may constitute one trial of a TMT, in particular of a TMT-A. More in particular, the screenshots 20c to 20e may show the first of two trials belonging to the TMT-A. The shown trial comprises the stimuli “1” to “15”, meanwhile a second (not shown) trial comprises the stimuli “16” to “30”.

In optional screenshot 20a, a test person or another user may start the test, e.g. by tapping or clicking a start button. Once the test has been started, a counter may be run in optional screenshot 20b. Accordingly, the test person is prepared to respond in a timely manner to the stimuli presented in the test (cf. from screenshot 20c on). It is namely desirable that the test person responds as quickly and as accurately as possible, in order to obtain objective test results. This may be advantageous, as the time to successfully complete the test may be used to calculate a test result. Hence, the test person should desirably be enabled to start at each run in the same way without any delays.

In screenshot 20c a first screenshot of the test is shown. In particular, screenshot 20c shows the beginning of the test. In the test the test person is expected to select the stimuli according to their predefined order, such that the response to a stimulus is a function of the response to the preceding stimulus according to the predefined order. In the present example, the test person is expected to consecutively select the stimuli “1” to “15”, i.e. first “1”, then “2”, then “3”, and so on. The stimuli may be presented as buttons on the screen which may be selected by tapping or clicking on them.

In order to assist the test person in the beginning, the first stimulus 14 (in the shown example stimulus “1”) may be preselected and/or highlighted (in a third predefined form, different to the first and second forms described below, e.g. by a highlighting the contour of the button).

Screenshot 20d shows an example screen during the test. In particular, it is shown the example where the test person has correctly selected a stimulus (in the present example stimulus “2”) in a preceding stimulus response. Accordingly, said stimulus “2” being the most recently (i.e. the preceding) correctly selected stimulus 15 is highlighted in a first predefined form, for example by highlighting the button of said stimulus “2” in a moderate form, e.g. by marking it in grey. This may assist the test person in memorizing how far he/she has already advanced in the current test.

Screenshot 20e shows a further example screen during the test. In this example the test person has selected an incorrect stimulus 16b in a preceding stimulus response, in the present example the stimulus “13”. Accordingly, the incorrectly selected stimulus may be highlighted in a second predefined form, for example by highlighting the button of said stimulus “13” in a more visible (i.e. more attracting the test person's gaze) form, e.g. by marking it in a color like red. In addition or alternatively, the highlighting in the second predefined form may comprise highlighting the frame of the pattern or of the complete screen, e.g. by marking it in a color like red. At the same time, the most recently (i.e. the preceding) correctly selected stimulus 15 (here stimulus “4”) may be highlighted in the first predefined form. Anyway, the system may be configured to allow the test person to retry, e.g. until one of the above-mentioned termination conditions is fulfilled (for example three times selecting a wrong stimulus).

FIG. 5 shows exemplary screenshots of a second test (here test-type TMT-B) implemented on a smartphone or another electronic device. The test shown in FIG. 5, in particular in screenshots 30c to 30e, may constitute one trial of a TMT, in particular of a TMT-B. More in particular, the screenshots 30c to 30e may show the first of two trials belonging to the TMT-B. TMT-B may comprise ascending numbers and letters in an alternating order, i.e. 1-A-2-B-3-C etc. The shown trial comprises the stimuli “1” to “8” (i.e. 15 stimuli in total), meanwhile a second (not shown) trial may comprise e.g. the stimuli “H” to “O” (e.g. 15 stimuli in total). Beside this difference the exemplary test of FIG. 5 may substantially correspond to that one of FIG. 4.

However, it is noted that according to this example not only the most recently (i.e. the preceding) correctly selected stimulus 15a may be highlighted, but also the second most recently correctly selected stimulus 15b. For example, stimulus 15a may be highlighted in a more visible form than stimulus 15b. In this way, the test person may be assisted in remembering how far he/she has already advanced in the current test, with regard to both types of stimuli, i.e. number and letters.

Further implementations are summarized in the following examples.

Example 1: A Computer-implemented method of providing a plurality of consecutive tests for a test person, the method comprising the steps of: providing at least one set of visual stimuli having a predefined order, wherein in a test the test person is expected to select the stimuli according to their predefined order, such that the response to a stimulus is a function of the response to the preceding stimulus according to the predefined order, providing a pattern defined by a plurality of predetermined positions, for each of the plurality of tests, mapping the stimuli to the pattern in a unique sequence according to a pseudo-random distribution function, wherein said function is defined: such that a mean distance between each pair of consecutive pattern positions, to which a stimulus and its preceding stimulus are mapped, is within a first predetermined range for each of the plurality of tests, and/or such that a total distance is within a second predetermined range for each of the plurality of tests, the total distance summing the distances between each pair of consecutive pattern positions to which a stimulus and its preceding stimulus are mapped.

Example 2: The method according to example 1, wherein the pattern has a grid form, and/or the distance between each pair of consecutive pattern positions, to which a stimulus and its preceding stimulus are mapped in the pattern having a grid form, is defined by the number of horizontal and/or vertical steps in the grid between the pair.

Example 3 The method according to example 1 or 2, wherein the mean distance is an arithmetic mean step count.

Example 4: The method according to any one of the preceding examples, wherein the pseudo-random distribution function comprises at least one rule which is defined such that clusters of consecutive stimuli on the pattern are reduced.

Example 5: The method according to any one of the preceding examples, wherein the stimuli are mapped to the pattern in a pseudo-randomized sequence, and/or the same pseudo-random distribution function is used for all tests, and/or the pattern is identical for all tests, and/or the stimuli comprise numerical or alphanumerical stimuli or other signs.

Example 6: The method according to any one of the preceding examples, wherein the number of positions of the pattern corresponds to the number of stimuli of one set of stimuli.

Example 7: The method according to any one of the preceding examples, wherein an individual test comprises two consecutive test parts, and at least one set of stimuli comprises a first set for the first test part and a second set for the test second part, the stimuli of the second set following the stimuli of the first set according to the predefined order of the first set, wherein in the first test part the first set is mapped to the positions of the pattern, and consecutively in the second test part the second set is mapped to the positions of the pattern.

Example 8: The method according to any one of the preceding examples, wherein the plurality of tests comprises a first and a second type of test which are provided alternately, wherein the at least one set of stimuli comprises a first set or a first and second set for the first type of test and a third set or a third and fourth set for the second type of test.

Example 9: The method according to the preceding example, wherein the pseudo-random distribution function is defined such that the mean distance of consecutive stimuli within a couple of first and second types of tests on the pattern is the same or within a third predetermined range being smaller than the first predetermined range.

Example 10: The method according to any one of the preceding examples, further comprising the step of: for each individual test presenting the stimuli mapped to the positions of the pattern on a display or a touchscreen.

Example 11: The method according to any one of the preceding examples, further comprising the step of: calculating a test result as a function of the test person's stimulus responses, and/or as a function of the test person's gestures in response to the presented stimuli.

Example 12: The method according to the preceding example, wherein the stimulus responses are tap responses on the touchscreen for selecting a stimulus.

Example 13: The method according to any one of the preceding examples 11-12, wherein the test result is calculated as a function of whether the test person has selected the stimuli according to their correct predefined order and/or as a function of the test person's response time.

Example 14: The method according to any one of the preceding examples 11-13, further comprising the steps of: highlighting the most recently correctly selected stimulus in a first predefined form, and/or in case the test person selects an incorrect stimulus, highlighting the incorrectly selected stimulus in a second predefined form.

Example 15: A computer program comprising computer-readable instructions which when executed by a data processing system cause the data processing system to carry out the method according to any one of preceding method examples.

Example 16: A recording medium readable by a computer and having recorded thereon a computer program including instructions for executing the steps of a method according to any one of the preceding method examples.

Example 17: A processing device for providing a plurality of consecutive tests for a test person, configured to: provide at least one set of visual stimuli having a predefined order, wherein in a test the test person is expected to select the stimuli according to their predefined order, such that the response to a stimulus is a function of the response to the preceding stimulus according to the predefined order, provide a pattern defined by a plurality of predetermined positions, for each of the plurality of tests, map the stimuli to the pattern in a unique sequence according to a pseudo-random distribution function, wherein said function is defined: such that a mean distance between each pair of consecutive pattern positions, to which a stimulus and its preceding stimulus are mapped, is within a first predetermined range for each of the plurality of tests, and/or such that a total distance is within a second predetermined range for each of the plurality of tests, the total distance summing the distances between each pair of consecutive pattern positions to which a stimulus and its preceding stimulus are mapped.

In this specification the phrase “configured to” is used in different contexts related to computer systems, hardware, or part of a computer program. When a system is said to be configured to perform one or more operations, this means that the system has appropriate software, firmware, and/or hardware installed on the system that, when in operation, causes the system to perform the one or more operations. When some hardware is said to be configured to perform one or more operations, this means that the hardware includes one or more circuits that, when in operation, receive input and generate output according to the input and corresponding to the one or more operations. When a computer program is said to be configured to perform one or more operations, this means that the computer program includes one or more program instructions, that when executed by one or more computers, causes the one or more computers to perform the one or more operations.

Unless otherwise stated, the foregoing alternative examples are not mutually exclusive, but may be implemented in various combinations to achieve unique advantages. In the foregoing description, the provision of the examples described, as well as clauses phrased as “such as,” “including” and the like, should not be interpreted as limiting embodiments to the specific examples; rather, the examples are intended to illustrate only one of many possible embodiments.

1. A computer-implemented method of providing a plurality of consecutive tests for a test person,
the method comprising the steps of:
providing at least one set of visual stimuli having a predefined order, wherein in a test the test person is expected to select the stimuli according to their predefined order, such that the response to a stimulus is a function of the response to the preceding stimulus according to the predefined order,
providing a pattern defined by a plurality of predetermined positions,
for each of the plurality of tests, mapping the stimuli to the pattern in a unique sequence according to a pseudo-random distribution function,
wherein said function is defined:
such that a mean distance between each pair of consecutive pattern positions, to which a stimulus and its preceding stimulus are mapped, is within a first predetermined range for each of the plurality of tests, and/or
such that a total distance is within a second predetermined range for each of the plurality of tests, the total distance summing the distances between each pair of consecutive pattern positions to which a stimulus and its preceding stimulus are mapped.

2. The method according to claim 1, wherein
the pattern has a grid form, and/or
the distance between each pair of consecutive pattern positions, to which a stimulus and its preceding stimulus are mapped in the pattern having a grid form, is defined by the number of horizontal and/or vertical steps in the grid between the pair.

3. The method according to claim 1, wherein
the mean distance is an arithmetic mean step count.

4. The method according to claim 1, wherein
the pseudo-random distribution function comprises at least one rule which is defined such that clusters of consecutive stimuli on the pattern are reduced.

5. The method according to claim 1, wherein
the stimuli are mapped to the pattern in a pseudo-randomized sequence, and/or the same pseudo-random distribution function is used for all tests, and/or
the pattern is identical for all tests, and/or
the stimuli comprise numerical or alphanumerical stimuli or other signs.

6. The method according to claim 1, wherein
the number of positions of the pattern corresponds to the number of stimuli of one set of stimuli.

7. The method according to claim 1, wherein
an individual test comprises two consecutive test parts, and
at least one set of stimuli comprises a first set for the first test part and a second set for the test second part, the stimuli of the second set following the stimuli of the first set according to the predefined order of the first set, wherein
in the first test part the first set is mapped to the positions of the pattern, and consecutively in the second test part the second set is mapped to the positions of the pattern.

8. The method according to claim 1, wherein
the plurality of tests comprises a first and a second type of test which are provided alternately, wherein
the at least one set of stimuli comprises a first set or a first and second set for the first type of test and a third set or a third and fourth set for the second type of test.

9. The method according to claim 8, wherein
the pseudo-random distribution function is defined such that the mean distance of consecutive stimuli within a couple of first and second types of tests on the pattern is the same or within a third predetermined range being smaller than the first predetermined range.

10. The method according to claim 1, further comprising the step of:
for each individual test presenting the stimuli mapped to the positions of the pattern on a display or a touchscreen.

11. The method according to claim 1, further comprising the step of:
calculating a test result as a function of the test person's stimulus responses, and/or as a function of the test person's gestures in response to the presented stimuli.

12. The method according to claim 11, wherein
the stimulus responses are tap responses on the touchscreen for selecting a stimulus.

13. The method according to claim 11, wherein
the test result is calculated as a function of whether the test person has selected the stimuli according to their correct predefined order and/or as a function of the test person's response time.

14. The method according to claim 11, further comprising the steps of:
highlighting the most recently correctly selected stimulus in a first predefined form, and/or in case the test person selects an incorrect stimulus, highlighting the incorrectly selected stimulus in a second predefined form.

15. A computer program comprising computer-readable instructions which when executed by a data processing system cause the data processing system to carry out the method according to claim 1.

16. A recording medium readable by a computer and having recorded thereon a computer program including instructions for executing the steps of a method according to claim 1.

17. A processing device for providing a plurality of consecutive tests for a test person, configured to:
provide at least one set of visual stimuli having a predefined order, wherein in a test the test person is expected to select the stimuli according to their predefined order, such that the response to a stimulus is a function of the response to the preceding stimulus according to the predefined order,
provide a pattern defined by a plurality of predetermined positions,
for each of the plurality of tests, map the stimuli to the pattern in a unique sequence according to a pseudo-random distribution function,
wherein said function is defined:
such that a mean distance between each pair of consecutive pattern positions, to which a stimulus and its preceding stimulus are mapped, is within a first predetermined range for each of the plurality of tests, and/or
such that a total distance is within a second predetermined range for each of the plurality of tests, the total distance summing the distances between each pair of consecutive pattern positions to which a stimulus and its preceding stimulus are mapped.
